(19) United States
(12) Patent Application Publication
(10) Pub. No.: US 2026/0295118 A1
(43) Pub. Date: Oct. 1, 2026

(54) BIOLOGICAL MIXTURE COMPRISING BIOLOGICAL CROSS-LINKED MATERIAL, METHOD FOR SKIN TISSUE FILLING USING THE SAME AND PREPARATION METHOD THEREOF

(71) Applicant: Xeader Technology Co., Ltd., New Taipei City (TW)

(72) Inventors: PO YANG CHEN, New Taipei City (TW); JUN JIE WANG, New Taipei City (TW)

(73) Assignee: Xeader Technology Co., Ltd., New Taipei City (TW)

(21) Appl. No.: 19/630,072

(22) Filed: Mar. 26, 2026

(30) Foreign Application Priority Data
Mar. 31, 2025 (TW) 114112327

(51) Int. Cl. A61L 27/24; A61L 27/20; A61L 27/36

(52) U.S. Cl. CPC A61L 27/24; A61L 27/20; A61L 27/36

(57) ABSTRACT

The present invention provides a biological mixture, comprising a biological cross-linked material, and the biological cross-linked material comprises collagen, collagen peptide, a first hyaluronic acid and a second hyaluronic acid; wherein the collagen, the collagen peptide, the first hyaluronic acid and the second hyaluronic acid are cross-linked with each other. The biological mixture demonstrates a high degree of cross-linking, low collagen degradation rate and low hyaluronic acid degradation rate to increase the residence time thereof in the human body. The present invention further provides a method for skin tissue filling by providing the biological mixture and a preparation method for the biological mixture.

### CROSS-REFERENCE TO RELATED APPLICATION

Pursuant to 35 U.S.C. § 119(a), this application claims the benefits of the priority to Taiwan Patent Application No. 114112327, filed on Mar. 31, 2025, which is incorporated by reference herein by its entirety.

### BACKGROUND OF THE INVENTION

### 1. Field of the Invention

The present invention relates to a biological mixture, especially the biological mixture comprising a biological cross-linked material. The present invention further relates to a method for skin tissue filling by providing the biological mixture, and a preparation method for the biological mixture.

### 2. Description of the Prior Arts

Medical cosmetology gradually emerges along with people's frequent social activities, and comprises facial care, body contouring and hair care, etc. As face serves as the primary visual impression of individuals, how to effectively maintain the young look and health of the face becomes the top aims for medical cosmetology.

There are various medical skin care products currently available, such as hyaluronic acid injections and collagen injections, which are injectable dermal fillers. When the superficial layer of skin becomes loose, wrinkled or sunken resulting from aging, subcutaneous injections of hyaluronic acid or collagen can be used for skin appearance adjustment or contouring. However, both hyaluronic acid and collagen degrade easily after injection, and require regular supplemental injection, so how to effectively reduce the degradation time of such materials in the human body is an important issue.

### SUMMARY OF THE INVENTION

To solve the aforementioned issue, the present invention provides a biological mixture, comprising a biological cross-linked material, and the biological cross-linked material comprises collagen, collagen peptide, a first hyaluronic acid and a second hyaluronic acid; wherein the first hyaluronic acid has a molecular weight of 80 kilodaltons (kDa) to 500 kDa, and the second hyaluronic acid has a molecular weight of 1000 kDa to 3000 kDa; the collagen, the collagen peptide, the first hyaluronic acid and the second hyaluronic acid are cross-linked with each other; and the biological cross-linked material has an average degree of cross-linking from 65% to 97%.

According to the present invention, the biological cross-linked material or the biological mixture has an excellent structural strength, such as high degree of cross-linking, low collagen degradation rate and low hyaluronic acid degradation rate, so as to increase the residence time of the biological cross-linked material (comprising collagen, collagen peptide, a first hyaluronic acid and a second hyaluronic acid) in the human body, and the frequency of periodical supplementation of hyaluronic acid and/or collagen can be effectively reduced.

In one embodiment, the biological mixture comprises water. The biological mixture can be freeze-dried to obtain the biological cross-linked material. The biological cross-linked material of the present invention can be prepared into a form of a lyophilized powder for long term storage, and be rehydrated before use, for example, adding water or a pharmaceutically acceptable carrier, such as phosphate buffered saline (PBS).

For clarification, the collagen has a main structure of a repeated amino acid sequence comprising (glycine)-(hydroxylysine)-(hydroxyproline), but not limited thereto. Preferably, the collagen has a main structure of a repeated amino acid sequence composed of (glycine)-(hydroxylysine)-(hydroxyproline), but not limited thereto. Further, the collagen peptide has ingredients that are rich in glycine, hydroxylysine and hydroxyproline, but not limited thereto. As collagen, collagen peptide, the first hyaluronic acid and the second hyaluronic acid each are not a single ingredient or a compound with a single molecular weight, the biological mixture comprises the biological cross-linked material obtained by cross-linking such 4 types of ingredients by means of various bonding methods.

In one embodiment, the collagen comprises a repeated sequence of (glycine)-(hydroxylysine)-(hydroxyproline).

In one embodiment, the collagen comprises type I collagen.

In one embodiment, the collagen peptide comprises glycine, hydroxylysine, hydroxyproline or a combination thereof.

In one embodiment, the collagen peptide is a degredation product obtained by using pepsin. Preferably, the collagen peptide is a degredation product obtained by using pepsin to digest type I collagen.

Preferably, the first hyaluronic acid has a molecular weight of 85 kDa to 450 kDa, such as 85 kDa, 100 kDa, 150 kDa, 200 kDa, 250 kDa, 300 kDa, 350 kDa, 400 kDa or 450 kDa. More preferably, the first hyaluronic acid has a molecular weight of 90 kDa to 110 kDa.

Preferably, the second hyaluronic acid has a molecular weight of 1100 kDa to 2900 kDa, such as 1100 kDa, 1400 kDa, 1700 kDa, 2000 kDa, 2300 kDa, 2600 kDa or 2900 kDa. More preferably, the first hyaluronic acid has a molecular weight of 1350 kDa to 1650 kDa.

Preferably, the molecular weight may be an average molecular weight. More preferably, the average molecular weight may be a weight-average molecular weight (Mw) or a number-average molecular weight (Mn).

Preferably, the biological cross-linked material has an average degree of cross-linking from 66% to 96%, such as 66%, 70%, 75%, 80%, 85%, 90%, 95% or 96%. Preferably, the biological cross-linked material has an average degree of cross-linking from 72% to 78%.

In one embodiment, an average collagen degradation rate of the biological cross-linked material is less than 20%. Preferably, the average collagen degradation rate of the biological cross-linked material is less than 15%. More preferably, the average collagen degradation rate of the biological cross-linked material is 3% to 10%.

In one embodiment, an average hyaluronic acid degradation rate of the biological cross-linked material is less than 17%. Preferably, the average hyaluronic acid degradation rate of the biological cross-linked material is less than 15%. More preferably, the average hyaluronic acid degradation rate of the biological cross-linked material is 9% to 14%.

In one embodiment, the biological mixture and the biological cross-linked material are substantially the same, that is, the average degree of cross-linking, the average collagen degradation rate and/or the average hyaluronic acid degradation rate of the biological cross-linked material are those of the biological mixture.

In one embodiment, based on a total volume of the biological cross-linked material, the collagen and the collagen peptide are in a total amount of 30 mg/ml to 80 mg/ml, such as 30 mg/ml, 40 mg/ml, 50 mg/ml, 60 mg/ml, 70 mg/ml or 80 mg/ml. Preferably, based on a total volume of the biological cross-linked material, the collagen and the collagen peptide are in a total amount of 35 mg/ml to 45 mg/ml.

In one embodiment, based on a total volume of the biological cross-linked material, the first hyaluronic acid and the second hyaluronic acid are in a total amount of 1 mg/ml to 18 mg/ml, such as 1 mg/ml, 3 mg/ml, 6 mg/ml, 9 mg/ml, 12 mg/ml, 15 mg/ml or 18 mg/ml. Preferably, based on a total volume of the biological cross-linked material, the first hyaluronic acid and the second hyaluronic acid are in a total amount of 4.5 mg/ml to 5.5 mg/ml.

In one embodiment, the biological mixture further comprises exosomes. Preferably, the exosomes comprise adipose-derived stem cell exosomes.

In one embodiment, based on a total volume of the biological mixture, the exosomes have a concentration of 1×10⁵ extracellular vesicles (EV)/ml to 1×10¹¹ EV/ml, such as 1×10⁵ EV/ml, 5×10⁵ EV/ml, 1×10⁶ EV/ml, 5×10⁶ EV/ml, 1×10⁷ EV/ml, 5×10⁷ EV/ml, 1×10⁸ EV/ml, 5×10⁸ EV/ml, 1×10⁹ EV/ml, 5×10⁹ EV/ml, 1×10¹⁰ EV/ml, 5×10¹⁰ EV/ml or Ix 10¹¹ EV/ml. Preferably, based on a total volume of the biological mixture, the exosomes have a concentration of 8×10⁶ EV/ml to 1.2×10⁷ EV/ml.

The present invention further provides a method for skin tissue filling, comprising providing the biological mixture into a skin.

The present invention also provides a method for skin tissue filling, comprising administering to a subject in need thereof an effective amount of the biological mixture into a skin.

In one embodiment, the biological mixture is in an injection dosage form. Preferably, the biological mixture is an injectable dermal filler. More preferably, the biological mixture is an injectable dermal filler for a micro-plastic surgery.

In one embodiment, the method for skin tissue filling of the present invention provides the biological mixture or the biological cross-linked material into the skin by means of a subcutaneous injection.

Preferably, the method for skin tissue filling is a method for skin tissue filling for face.

In one embodiment, the biological mixture can be a drug, a cosmetic product or a medical device. Preferably, the biological mixture can be a cosmetic drug, a medical cosmetic product or a cosmetic medical device.

The present invention further provides a preparation method for the biological mixture, comprising:
a first cross-linking step: mixing the collagen, the first hyaluronic acid, a first cross-linking agent and a first carrier to obtain a first mixture for carrying out a first cross-linking reaction for 20 hours to 28 hours to obtain a first cross-linked complex; wherein based on a total volume of the first mixture, the collagen is in an amount of 10 weight/volume percent (w/v %) to 40 w/v %, and the first hyaluronic acid is in an amount of 0.1 w/v % to 9 w/v %;
a second cross-linking step: mixing the first cross-linked complex, the second hyaluronic acid, a second cross-linking agent and a second carrier to obtain a second mixture for carrying out a second cross-linking reaction for 20 hours to 28 hours to obtain a second cross-linked complex; wherein based on a total volume of the second mixture, the first cross-linked complex is in an amount of 10 w/v % to 40 w/v %, and the second hyaluronic acid is in an amount of 0.1 w/v % to 9 w/v %; and
a third cross-linking step: mixing the second cross-linked complex, the collagen peptide, a third cross-linking agent and a third carrier to obtain a third mixture for carrying out a third cross-linking reaction for 20 hours to 28 hours to obtain the biological cross-linked material; wherein based on a total volume of the third mixture, the second cross-linked complex is in an amount of 10 w/v % to 40 w/v %, and the collagen peptide is in an amount of 10 w/v % to 40 w/v %; wherein
the first cross-linking agent, the second cross-linking agent and the third cross-linking agent are different from each other, and the first carrier, the second carrier and the third carrier each comprise water.

Preferably, the first carrier, the second carrier, the third carrier or a combination thereof comprises phosphate buffered saline (PBS).

In one embodiment, the collagen is obtained from a collagen solution, and the collagen in the collagen solution is in a concentration of 10 mg/ml to 16 mg/ml, such as 10 mg/ml, 11 mg/ml, 12 mg/ml, 13 mg/ml, 14 mg/ml, 15 mg/ml or 16 mg/ml. Preferably, the collagen in the collagen solution is in a concentration of 10.5 mg/ml to 11.5 mg/ml.

In one embodiment, the collagen peptide is obtained from a collagen peptide solution, and the collagen peptide in the collagen peptide solution is in a concentration of 25 mg/ml to 50 mg/ml, such as 25 mg/ml, 30 mg/ml, 35 mg/ml, 40 mg/ml, 45 mg/ml or 50 mg/ml. Preferably, the collagen peptide in the collagen peptide solution is in a concentration of 26 mg/ml to 30 mg/ml.

Preferably, based on a total volume of the first mixture, the collagen is in an amount of 15 w/v % to 35 w/v %, such as 15 w/v %, 19 w/v %, 23 w/v %, 27 w/v %, 31 w/v % or 35 w/v %, and the first hyaluronic acid is in an amount of 0.2 w/v % to 8 w/v %, such as 0.2 w/v %, 0.5 w/v %, 1 w/v %, 3 w/v %, 6 w/v % or 8 w/v %. Preferably, based on a total volume of the first mixture, the collagen is in an amount of 18 w/v % to 22 w/v %, and the first hyaluronic acid is in an amount of 0.8 w/v % to 1.2 w/v %.

Preferably, based on a total volume of the second mixture, the first cross-linked complex is in an amount of 15 w/v % to 35 w/v %, such as 15 w/v %, 19 w/v %, 23 w/v %, 27 w/v %, 31 w/v % or 35 w/v %, and the second hyaluronic acid is in an amount of 0.2 w/v % to 8 w/v %, such as 0.2 w/v %, 0.5 w/v %, 1 w/v %, 3 w/v %, 6 w/v % or 8 w/v %. Preferably, based on a total volume of the second mixture, the first cross-linked complex is in an amount of 18 w/v % to 22 w/v %, and the second hyaluronic acid is in an amount of 0.8 w/v % to 1.2 w/v %.

Preferably, based on a total volume of the third mixture, the second cross-linked complex is in an amount of 15 w/v % to 35 w/v %, such as 15 w/v %, 19 w/v %, 23 w/v %, 27 w/v %, 31 w/v % or 35 w/v %, and the collagen peptide is in an amount of 15 w/v % to 35 w/v %, such as 15 w/v %, 19 w/v %, 23 w/v %, 27 w/v %, 31 w/v % or 35 w/v %. Preferably, based on a total volume of the third mixture, the second cross-linked complex is in an amount of 18 w/v % to 22 w/v %, and the collagen peptide is in an amount of 18 w/v % to 22 w/v %.

In one embodiment, a material source for the collagen in the collagen solution and/or the collagen peptide in the collagen peptide solution comprises skin of pig, cattle, goat, sheep or a combination thereof. Preferably, the material source comprises a skin of cattle. More preferably, the cattle comprise cow, bull, ox, steer, calf or a combination thereof.

In one embodiment, the collagen in the collagen solution comprises pig-derived collagen, cattle-derived collagen, goat-derived collagen, sheep-derived collagen or a combination thereof. Preferably, the collagen in the collagen solution comprises cattle-derived collagen.

In one embodiment, the first cross-linking agent comprises 1,4-butanediol diglycidal ether (BDDE).

In one embodiment, based on a total volume of the first mixture, the first cross-linking agent is in an amount of 0.5 w/v % to 5 w/v %, such as 0.5 w/v %, 1 w/v %, 1.5 w/v %, 2 w/v %, 2.5 w/v %, 3 w/v %, 3.5 w/v %, 4 w/v %, 4.5 w/v % or 5 w/v %. Preferably, based on a total volume of the first mixture, the first cross-linking agent is in an amount of 1.8 w/v % to 2.2 w/v %.

In one embodiment, the second cross-linking agent comprises glutaraldehyde (GA).

In one embodiment, based on a total volume of the second mixture, the second cross-linking agent is in an amount of 0.005 w/v % to 0.1 w/v %, such as 0.005 w/v %, 0.01 w/v %, 0.04 w/v %, 0.06 w/v %, 0.08 w/v % or 0.1 w/v %. Preferably, based on a total volume of the second mixture, the second cross-linking agent is in an amount of 0.009 w/v % to 0.011 w/v %.

In one embodiment, the third cross-linking agent comprises 1-ethyl-3-[3-dimethylaminopropyl]carbodiimide (EDC).

In one embodiment, based on a total volume of the third mixture, the third cross-linking agent is in an amount of 0.5 w/v % to 5 w/v %, such as 0.5 w/v %, 1 w/v %, 1.5 w/v %, 2 w/v %, 2.5 w/v %, 3 w/v %, 3.5 w/v %, 4 w/v %, 4.5 w/v % or 5 w/v %. Preferably, based on a total volume of the third mixture, the third cross-linking agent is in an amount of 2.7 w/v % to 3.3 w/v %.

In one embodiment, the first cross-linking step and/or the third cross-linking step comprise adding an alkaline agent. Preferably, the alkaline agent comprises sodium hydroxide.

In one embodiment, the alkaline agent is a pH adjuster.

In one embodiment, the first mixture in the first cross-linking step and/or the third mixture in the third cross-linking step or a combination thereof is alkaline. Preferably, the first mixture in the first cross-linking step and/or the third mixture in the third cross-linking step has a pH value of 8 to 12, such as 8, 9, 10, 11 or 12. More preferably, the first mixture in the first cross-linking step and/or the third mixture in the third cross-linking step has a pH value of 9.8 to 10.2.

In one embodiment, the first cross-linking step, the second cross-linking step, the third cross-linking step or a combination thereof comprises a precipitation step, so that a sediment can be obtained. For example, a first sediment can be obtained after a first precipitation step in the first cross-linking step; a second sediment can be obtained after a second precipitation step in the second cross-linking step; and/or a third sediment can be obtained after a third precipitation step in the third cross-linking step. Preferably, the precipitation step comprises adding a sodium chloride aqueous solution, and then carrying out a centrifugation at 10000×g to 15000×g for 5 minutes to 20 minutes. More preferably, the precipitation step comprises adding a sodium chloride aqueous solution, and then carrying out a centrifugation at 12000×g to 13000×g for 8 minutes to 12 minutes. The respective sediment mainly comprises the corresponding cross-linked complex in the respective cross-linking step. That is, the first sediment mainly comprises the first cross-linked complex, the second sediment mainly comprises the second cross-linked complex, and/or the third sediment mainly comprises the biological cross-linked material.

In one embodiment, the first cross-linking step, the second cross-linking step, the third cross-linking step or a combination thereof comprises a cleaning step, so that a cleaned sediment can be obtained. For example, the first sediment is subjected to a first cleaning step in the first cross-linking step to obtain the first cross-linked complex; the second sediment is subjected to a second cleaning step in the second cross-linking step to obtain the second cross-linked complex; and/or the third sediment is subjected to a third cleaning step in the third cross-linking step to obtain the biological cross-linked material. Preferably, the cleaning step comprises adding an ethanol aqueous solution, and then carrying out a centrifugation at 10000×g to 15000×g for 5 minutes to 20 minutes. More preferably, the cleaning step comprises adding an ethanol aqueous solution, and then carrying out a centrifugation at 12000×g to 13000×g for 8 minutes to 12 minutes.

In one embodiment, the preparation method for the biological mixture further comprises a cross-linking neutralization step: mixing the biological cross-linked material and a glycine aqueous solution to obtain a fourth mixture, and the fourth mixture is subjected to a cross-linking neutralization reaction for 20 hours to 28 hours to obtain a biological cross-linked material product. According to the present invention, the cross-linking neutralization step can ensure that the cross-linking reaction is not carried out for the biological cross-linked material product, so as to improve product stability. Further, there is no substantial weight difference between the biological cross-linked material and the biological cross-linked material product, or the weight difference thereof is within 1%.

Preferably, the glycine aqueous solution is in a concentration of 0.05 M to 1 M. Preferably, the glycine aqueous solution is in a concentration of 0.08 M to 0.12 M.

In one embodiment, the cross-linking neutralization step comprises a fourth precipitation step, so that a sediment can be obtained. That is, a fourth sediment can be obtained after the cross-linking neutralization step. Preferably, the fourth precipitation step comprises carrying out a centrifugation at 10000×g to 15000×g for 1 minute to 10 minutes. More preferably, the fourth precipitation step comprises carrying out a centrifugation at 12000×g to 13000×g for 2.5 minutes to 3.5 minutes.

In one embodiment, the cross-linking neutralization step comprises a fourth cleaning step, so that a cleaned sediment can be obtained. That is, the fourth sediment is subjected to the fourth cleaning step in the cross-linking neutralization step to obtain the biological cross-linked material product. Preferably, the fourth cleaning step comprises adding phosphate buffered saline, and then carrying out a centrifugation at 10000×g to 15000×g for 1 minute to 10 minutes. More preferably, the fourth cleaning step comprises adding phosphate buffered saline, and then carrying out a centrifugation at 12000×g to 13000×g for 2.5 minutes to 3.5 minutes.

To sum up, the biological mixture comprising the biological cross-linked material of the present invention is a biomaterial for tissue supplementation and repair in a bionic repair approach, and is suitable for subcutaneous tissue filling, wrinkles reduction and medical applications such as tissue repair, scar repair and tissue reconstruction, etc. Further, the biological mixture of the present invention has good biocompatibility and can reduce the risk of allergic or inflammatory reactions. Finally, the combination of the biological cross-linked material and exosomes can accelerate tissue repair to further promote human tissue health.

### BRIEF DESCRIPTION OF THE DRAWINGS

FIG. 1 is a flowchart showing one embodiment of a preparation method for the biological mixture of the present invention.

FIG. 2 is a flowchart showing another embodiment of a preparation method for the biological mixture of the present invention.

FIG. 3 is the picture of the biological mixture of the present invention in an injection dosage form.

### DETAILED DESCRIPTION OF THE PREFERRED EMBODIMENTS

### I. Raw Materials

### (I) Collagen Solution

1. Steps: 6 grams (g) of pig skin fragments (size: 1 cm×1 cm×0.04 cm) and 600 mg pepsin were put in a 400 ml 0.01 N aqueous hydrochloric acid solution, then stirred at room temperature for 12 hours with an electromagnetic stirring bar (rotation speed: 250 rpm) to carry out the digestion reaction, and filtered with a square filter cloth (a steamer Mesh Cloth, about 1800 to 1900 mesh) to obtain a collagen solution.
2. Analysis: This analysis measures the concentration of the collagen solution. Hydroxyproline (Hypro) is mostly found only in connective tissue scleroprotein, collagen and elastin, and the primary structure of collagen is mostly a repeated amino sequence of (glycine)-(hydroxylysine)-(hydroxyproline), so the well-recognized quantification method for collagen is to measure the content of hydroxyproline. The present invention refers to the method recited in “A. L. Helling et al., In Vitro Enzymatic Degradation of Tissue Grafts and Collagen Biomaterials by Matrix Metalloproteinases: Improving the Collagenase Assay, ACS Biomaterials Science & Engineering Vol 3/Issue 9, 2016” to measure the concentration of hydroxyproline, and abbreviates as follows:
(1) Reference standard: this experiment diluted the reference standard of hydroxyproline to obtain hydroxyproline solutions with different concentrations, and used spectrophotometer to measure the absorbance value at a wavelength of 555 nanometers (nm) to draw a standard curve of absorbance value versus the concentration of hydroxyproline.
(2) Collagen solution: the collagen solution was subjected to freeze-drying to obtain a lyophilized powder, and further subjected to a hydrolysis reaction with concentrated hydrochloric acid at 100° C. for 16 hours to obtain a hydrolyzate. The hydrolyzate was diluted 50 times with distilled water, and then further diluted with isopropanol to obtain a diluted solution, wherein the addition amount of isopropyl alcohol was 2 times that of distilled water to obtain a diluted solution. The diluted solution was added with chloramine T reagent, citrate buffer, distilled water, Ehrlich's reagent, perchloric acid and isopropanol, then homogenized, and incubated at 70° C. for 10 minutes. The spectrophotometer was used to measure the absorbance value at a wavelength of 555 nm, and match the results with the standard curve to obtain the concentration of hydroxyproline in the collagen solution.

Further, the article “N. Yu. Ignat'eva et al., Determination of Hydroxyproline in Tissues and the Evaluation of the Collagen Content of the Tissues, Journal of Analytical Chemistry 62(1):51-57, 2007” disclosed that the proportion of hydroxyproline is generally 12.8% to 14.7% in different collagens, so this experiment uniformly assumed that the proportion of hydroxyproline is 13%. In other words, after the concentration of hydroxyproline is corrected by the dilution rate, the collagen content is 1/0.13=7.69 times that of hydroxypro line, and the calculation formula is as follows: Concentration of collagen (mg/ml)=(Concentration of hydroxyproline (mg/ml)×7.69).
3. Results: the concentration of the collagen in the collagen solution was about 11.1 mg/ml.

### (II) Collagen Peptide Solution

1. Steps: the steps to obtain the collagen solution and the collagen peptide solution were similar, and the only difference was that the digestion time is 72 hours for preparing the collagen peptide solution, not 12 hours. Further, due to the longer digestion time, the obtained collagen peptide solution mainly comprises collagen peptides resulting from the further digestion of collagen.
2. Analysis: the measuring and calculation methods for the concentration of the collagen peptide in the collagen peptide solution were the same with those of the concentration of the collagen in the collagen solution.
3. Results: the concentration of the collagen peptide in the collagen peptide solution was about 28.3 mg/ml.

### (III) First Hyaluronic Acid

Item name: HA-LM100, purchased from PASS BIOMED (Taiwan), the average molecular weight thereof was about 100 kDa.

### (IV) Second Hyaluronic Acid

Item name: HA-LM2000, purchased from PASS BIOMED (Taiwan), the average molecular weight thereof was about 1500 kDa.

### II. Example 1: The Biological Mixture Comprising Biological Cross-Linked Material

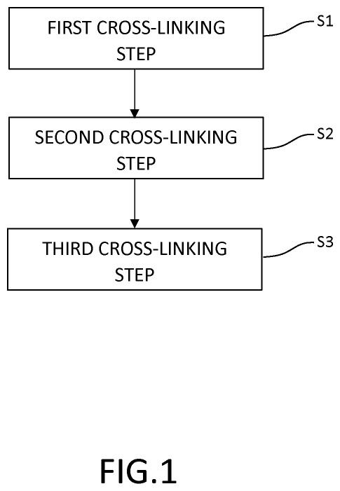

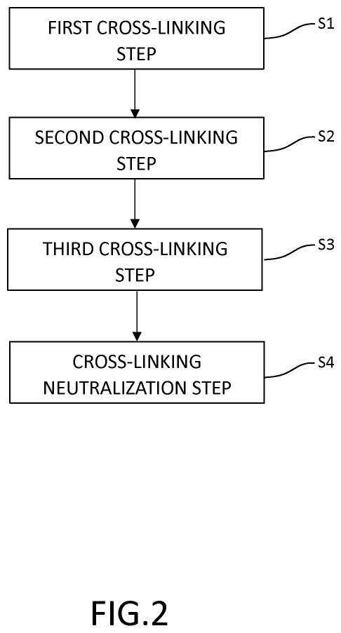

According to FIG. 1, the preparation method for the biological cross-linked material comprises: step (S1): first cross-linking step, step (S2): second cross-linking step and step (S3): third cross-linking step to obtain the biological mixture comprising the biological cross-linked material of the present invention. Further, according to FIG. 2, the biological cross-linked material of the present invention is further subjected to step (S4): cross-linking neutralization step to obtain the biological cross-linked material product. The details are as follows.

### (I) First Cross-Linking Step:

1. First mixing step: the collagen solution, the first hyaluronic acid, the first cross-linking agent (1,4-butanediol diglycidal ether, BDDE) and sodium hydroxide (NaOH) were added into phosphate buffered saline (PBS) to obtain a first mixture; wherein the collagen solution comprised collagen, and the first mixture had a pH=10. Based on a total volume of the first mixture, the collagen was in an amount of 20 w/v % (20 g/100 ml), the first hyaluronic acid was in an amount of 1 w/v %, the first cross-linking agent (BDDE) was in an amount of 2 w/v %, and the rest was phosphate buffered saline. For clarification, the collagen in an amount of 20 w/v % (20 g/100 ml) indicated that there was about 20 grams of collagen (which was equivalent to the weight of a lyophilized powder without water) per 100 ml of the first mixture. The amount of the first hyaluronic acid in said 1 w/v % also indicated the weight of the first hyaluronic acid per se without water.
2. First cross-linking step: the first mixture was stirred at a rotation speed of 250 rpm at room temperature (about 28° C.) for 24 hours to obtain the first cross-linking solution
3. First precipitation step: a sodium chloride (NaCl) aqueous solution in a concentration of 2M (2 mol/L) with the same volume as that of the first cross-linking solution was added to the first cross-linking solution, and then centrifugated at 12500×g for 10 minutes. The supernatant was removed to obtain a first sediment.
4. First cleaning step: a 60% ethanol aqueous solution in an appropriate volume was added to the first sediment, and then centrifugated at 12500×g for 10 minutes to remove the supernatant. After 5 times repeats, the first cross-linked complex was obtained; wherein the first cross-linked complex was a thick watery mixture. Further, the appropriate volume for the 60% ethanol aqueous solution indicated that 1 gram of the first sediment was added with about 1 ml of 60% ethanol aqueous solution.

### (II) Second Cross-Linking Step:

1. Second mixing step: the first cross-linked complex, the second hyaluronic acid and the second cross-linking agent (glutaraldehyde, GA) was added into PBS to obtain a second mixture; wherein the second mixture had a pH=7.2. Based on a total volume of the second mixture, the first cross-linked complex was in an amount of 20 w/v %, the second hyaluronic acid was in an amount of 1 w/v %, and the second cross-linking agent (GA) was in an amount of 0.01 w/v %, and the rest was PBS.
2. Second cross-linking step: the second mixture was stirred at a rotation speed of 250 rpm at room temperature for 24 hours to obtain the second cross-linking solution.
3. Second precipitation step: the second precipitation step was similar to the first precipitation step, the only difference was that the precipitation target was the second cross-linking solution, not the first cross-linking solution. In other words, a 2M sodium chloride aqueous solution with the same volume as that of the second cross-linking solution was added to the second cross-linking solution, and then centrifugated at 12500×g for 10 minutes. The supernatant was removed to obtain a second sediment.
4. Second cleaning step: the second cleaning step was similar to the first cleaning step, and the only differences were that the cleaning target was the second sediment, not the first sediment, and there were only 3 times repeats, not 5 times repeats. In other words, a 60% ethanol aqueous solution in an appropriate volume was added to the second sediment, and then centrifugated at 12500×g for 10 minutes to remove the supernatant. After 3 times repeats, the second cross-linked complex was obtained; wherein the second cross-linked complex was a thick watery mixture.

### (III) Third Cross-Linking Step:

1. Third mixing step: the second cross-linked complex, the collagen peptide solution, the third cross-linking agent (1-ethyl-3-[3-dimethylaminopropyl]carbodiimide, EDC) and sodium hydroxide were added into PBS to obtain a third mixture; wherein the third mixture had a pH=10. Based on a total volume of the third mixture, the second cross-linked complex was in an amount of 20 w/v %, the collagen peptide solution was in an amount of 20 w/v %, and the third cross-linking agent (EDC) was in an amount of 3 w/v %, and the rest was PBS.
2. Third cross-linking step: the third mixture was stirred at a rotation speed of 250 rpm at 40° C. for 24 hours to obtain the third cross-linking solution.
3. Third precipitation step: the third precipitation step was similar to the first precipitation step, the only difference was that the precipitation target was the third cross-linking solution, not the first cross-linking solution. The third sediment was obtained after the third precipitation step.
4. Third cleaning step: the third cleaning step was similar to the first cleaning step, the only differences were that the cleaning target was the third sediment, not the first sediment, and there were only 3 times repeats, not 5 times repeats. The biological cross-linked material obtained after the third cleaning step was a biological mixture and a thick watery mixture.

### (IV) Cross-Linking Neutralization Step:

1. Fourth mixing step: a 0.1 M glycine aqueous solution in a volume of times than that of the biological cross-linked material was added to the biological cross-linked material to obtain a fourth mixture.
2. Fourth cross-linking step: the fourth mixture was stirred at a rotation speed of 250 rpm at room temperature for 24 hours to obtain the fourth cross-linking solution.
3. Fourth precipitation step: the fourth cross-linking solution was centrifugated at 12500×g for 3 minutes, and the supernatant was removed to obtain a fourth sediment.
4. Fourth cleaning step: the fourth sediment was added with a mixture (pH=9) of PBS and sodium hydroxide in a volume the same as that of the fourth sediment, and then centrifugated at 12500×g for 3 minutes to remove the supernatant. After 3 times repeats, the biological cross-linked material product was obtained, which was a thick watery mixture. Further, there was no substantial difference in the wet weights between the biological cross-linked material product and the biological cross-linked material. For clarification, the cross-linking neutralization step is not a required step for preparing the biological cross-linked material of the present invention.

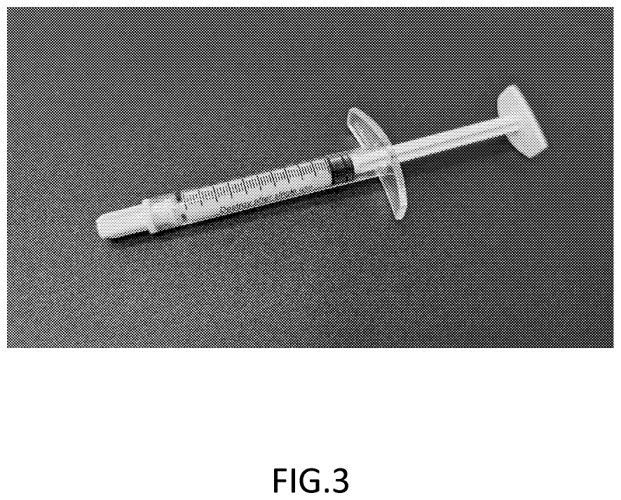

Further, Example 1 (the biological mixture comprising biological cross-linked material) was added to 0.35 w/v % lidocaine hydrochloride in PBS, and filled into a syringe to make an injection dosage form as shown in FIG. 3. Further, based on the total volume of the contents in the injection dosage form, the total concentration of collagen and collagen peptide was about 40 mg/ml.

### III. Monomer Ratio Analysis for the Biological Cross-Linked Material in Example 1

The monomers of the biological cross-linked material in Example 1 comprised the collagen from the collagen solution, the collagen peptide (resulting from collagen digestion) from the collagen peptide solution, the first hyaluronic acid and the second hyaluronic acid.

Concerning the quantification method for collagen and collagen peptide, the collagen and collagen peptide of the biological cross-linked material in Example 1 can be measured and calculated by the quantification method the same as that for the collagen solution, which comprised the measurement of hydroxyproline to calculate the concentrations of collagen and collagen peptide. The calculation result was 40 mg/ml, that is, the total concentration of collagen and collagen peptide of the biological cross-linked material in Example 1 was 40 mg/ml.

Concerning the quantification method for the first hyaluronic acid and the second hyaluronic acid, 1 ml of the biological cross-linked material of Example 1 was freeze-dried to obtain a lyophilized powder. The lyophilized powder was weighed to obtain a total weight, and the aforementioned amount of collagen and collagen peptide was subtracted from the total weight to obtain the amount of hyaluronic acid per ml, which was the total concentration of the first and second hyaluronic acids. The calculation result was 4.8 mg/ml, that is, the total concentration of the first hyaluronic acid and the second hyaluronic acid of the biological cross-linked material in Example 1 was 4.8 mg/ml.

For clarification, both the total concentrations of collagen and collagen peptide (40 mg/ml) and that of the first hyaluronic acid and the second hyaluronic acid (4.8 mg/ml) excluded the weight of water, so the total amount of 40+4.8=44.8 mg (pre ml) was not the wet weight of the biological cross-linked material (a thick watery mixture).

### IV. Example 2: The Combination of the Biological Cross-Linked Material and Exosomes

Example 1 (the biological mixture comprising biological cross-linked material) and an exosome solution (a concentrated adipose-derived stem cell exosome solution, item name: Exo-ASC, purchased from PASS BIOMED, Taiwan) were added to 0.35 w/v % lidocaine hydrochloride in PBS to obtain a fifth mixture. Based on a total volume of the fifth mixture, the biological cross-linked material was in an amount of 4 w/v %. Further, the adipose-derived stem cell exosomes in the fifth mixture were in a concentration of about 1×10⁷ EV/ml.

### V. Example 3 to Example 5: Biological Cross-Linked Material

Example 3 to Example 5 were similar to Example 1. The only difference was the source of collagen, elaborated as follows: the collagen raw materials for both the collagen solution and the collagen peptide solution in Example 1 were the skin (without hair) of pig. Those for both the collagen solution and the collagen peptide solution in Example 3 were the skins (without hair) of pig and cattle at a weight ratio of 1:1. Those for both the collagen solution and the collagen peptide solution in Example 4 were the skins (without hair) of pig and goat at a weight ratio of 1:1. Those for both the collagen solution and the collagen peptide solution in Example 5 were the skins (without hair) of cattle and goat at a weight ratio of 1:1. The collagen sources of all groups were shown in Table 1.

TABLE 1
the collagen raw materials for both the collagen solution
and the collagen peptide solution of all groups

| | Collagen raw materials for |
|---|---|
| | both thecollagen solution |
| Groups | and the collagen peptide solution |
| Example 1 | the skin of pig |
| Example 2 | the skin of pig |
| Example 3 | the skins of pig and cattle |
| | at a weight ratio of 1:1 |
| Example 4 | the skins of pig and goat |
| | at a weight ratio of 1:1 |
| Example 5 | the skins of cattle and |
| | goat at a weight ratio of 1:1 |

### VI. Comparative Example 1 (CE1) to Comparative Example 11 (CE11): Biological Cross-Linked Material (or a Combination with Exosomes)

The differences in the preparation methods of all groups were shown in Table 2, and the details were further elaborated in order as follows.

TABLE 2
the preparation method summary of all groups

| Groups | Preparation method summary |
|---|---|
| CE1 | 4 types of materials (collagen solution, collagen peptide solution, |
| | first hyaluronic acid and second hyaluronic acid) subjected to a |
| | single cross-linking step with 3 cross-linking agents (BDDE, GA |
| | and EDC); collagen source: skin of pig. |
| CE2 | 4 types of materials subjected to three cross-linking steps |
| | (similar to those for preparing Example 1) with the same cross- |
| | linking agent (BDDE); collagen source: skin of pig. |
| CE3 | 4 types of materials subjected to three cross-linking steps |
| | (similar to those for preparing Example 1) with the same cross- |
| | linking agent (GA); collagen source: skin of pig. |
| CE4 | 4 types of materials subjected to three cross-linking steps |
| | (similar to those for preparing Example 1) with the same cross- |
| | linking agent (EDC); collagen source: skin of pig. |
| CE5 | 2 types of materials (collagen solution and collagen peptide |
| | solution) subjected to a single cross-linking step with one cross- |
| | linking agent (GA); collagen source: skin of pig. |
| CE6 | 2 types of materials (first hyaluronic acid and second hyaluronic |
| | acid) subjected to a single cross-linking step with one cross- |
| | linking agent (BDDE). |
| CE7 | CE5 further added with exosomes; |
| | collagen source: skin of pig. |
| CE8 | CE6 further added with exosomes. |
| CE9 | Preparation method thereof was similar to that of CE3, and the |
| | only difference was the collagen source: the skins of pig and |
| | cattle at a weight ratio of 1:1 |
| CE10 | Preparation method thereof was similar to that of CE3, and the |
| | only difference was the collagen source: the skins of pig and goat |
| | at a weight ratio of 1:1 |
| CE11 | Preparation method thereof was similar to that of CE3, and the |
| | only difference was the collagen source: the skins of cattle and |
| | goat at a weight ratio of 1:1 |

(I) Comparative example 1: 4 types of materials (collagen solution, collagen peptide solution, first hyaluronic acid and second hyaluronic acid) were subjected to a single cross-linking step with 3 cross-linking agents (BDDE, GA and EDC). The details were as follows:

### 1. Cross-Linking Step:

(1) Mixing step: the collagen solution, the collagen peptide solution, the first hyaluronic acid, the second hyaluronic acid, the first cross-linking agent (BDDE), the second cross-linking agent (GA) and the third cross-linking agent (EDC) and sodium hydroxide were added into PBS to obtain a CE1 mixture; wherein the CE1 mixture had a pH=10. Based on a total volume of the CE1 mixture, the collagen solution was in an amount of 20 w/v %, the collagen peptide solution was in an amount of 20 w/v %, the first hyaluronic acid was in an amount of 1 w/v %, the second hyaluronic acid was in an amount of 1 w/v %, the first cross-linking agent (BDDE) was in an amount of 2 w/v %, the second cross-linking agent (GA) was in an amount of 0.01 w/v %, and third cross-linking agent (EDC) was in an amount of 3 w/v %. That is, the CE1 mixture was obtained after the mixing step.
(2) Cross-linking step: the CE1 mixture was stirred at a rotation speed of 250 rpm at room temperature for 24×3=72 hours to obtain a CE1 cross-linking solution.
(3) Precipitation step: the precipitation step in CE1 was similar to the first precipitation step in Example 1, and the only difference was that the precipitation target was the CE1 cross-linking solution to obtain a CE1 sediment.
(4) Cleaning step: the cleaning step in CE1 was similar to the first cleaning step in Example 1, and the only difference was that the cleaning target was the CE1 sediment to obtain a CE1 cross-linked complex.
2. Cross-linking neutralization step: the cross-linking neutralization step in CE1 was similar to that in Example 1, and the only difference was that the neutralization target was the CE1 cross-linked complex to obtain a CE1 cross-linked product.
(II) Comparative example 2: 4 types of materials (collagen solution, collagen peptide solution, first hyaluronic acid and second hyaluronic acid) were subjected to three cross-linking steps (similar to those for preparing Example 1) with the same cross-linking agent (BDDE). The details were as follows:
1. First cross-linking step: the first cross-linking step in CE2 was the same as that of Example 1.
2. Second cross-linking step: the second cross-linking step in CE2 was similar to that of Example 1, and the only differences were that the second cross-linking agent in CE2 was BDDE, not GA, and based on a total volume of the second mixture in CE2, the second cross-linking agent (BDDE) was in an amount of 2 w/v % to obtain a CE2 second cross-linked complex.
3. Third cross-linking step: the third cross-linking step in CE2 was similar to that of Example 1, and the only differences were that (1) the third cross-linking agent in CE2 was BDDE, not EDC, and based on a total volume of the third mixture in CE2, the third cross-linking agent (BDDE) was in an amount of 2 w/v %; and (2) the target in the third cross-linking step was the CE2 second cross-linked complex to obtain a CE2 cross-linked material.
4. Cross-linking neutralization step: the cross-linking neutralization step in CE2 was similar to that of Example 1, and the only difference was that the neutralization target was the CE2 cross-linked material to obtain a CE2 cross-linked product.
(III) Comparative example 3: 4 types of materials (collagen solution, collagen peptide solution, first hyaluronic acid and second hyaluronic acid) were subjected to three cross-linking steps (similar to those for preparing Example 1) with the same cross-linking agent (GA). The details were as follows:
1. First cross-linking step: the first cross-linking step in CE3 was similar to that of Example 1, and the only differences were that the first cross-linking agent in CE3 was GA, not BDDE, and based on a total volume of the first mixture in CE3, the first cross-linking agent (GA) was in an amount of 0.01 w/v % to obtain a CE3 first cross-linked complex.
2. Second cross-linking step: the second cross-linking step in CE3 was similar to that of Example 1, and the only difference was that the cross-linking target was the CE3 first cross-linked complex to obtain a CE3 second cross-linked complex.
3. Third cross-linking step: the third cross-linking step in CE3 was similar to that of Example 1, and the only differences were that (1) the third cross-linking agent in CE3 was GA, not EDC, and based on a total volume of the third mixture in CE3, the third cross-linking agent (GA) was in an amount of 0.01 w/v %; and (2) the target in the third cross-linking step was the CE3 second cross-linked complex to obtain a CE3 cross-linked material.
4. Cross-linking neutralization step: the cross-linking neutralization step in CE3 was similar to that of Example 1, and the only difference was that the neutralization target was the CE3 cross-linked material to obtain a CE3 cross-linked product.
(IV) Comparative example 4: 4 types of materials (collagen solution, collagen peptide solution, first hyaluronic acid and second hyaluronic acid) were subjected to three cross-linking steps (similar to those for preparing Example 1) with the same cross-linking agent (EDC). The details were as follows:
1. First cross-linking step: the first cross-linking step in CE4 was similar to that of Example 1, and the only differences were that the first cross-linking agent in CE4 was EDC, not BDDE, and based on a total volume of the first mixture in CE4, the first cross-linking agent (EDC) was in an amount of 3 w/v % to obtain a CE4 first cross-linked complex.
2. Second cross-linking step: the second cross-linking step in CE4 was similar to that of Example 1, and the only differences were that (1) the second cross-linking agent in CE4 was EDC, not GA, and based on a total volume of the second mixture in CE4, the second cross-linking agent (EDC) was in an amount of 3 w/v % to obtain a CE4 second cross-linked complex; and (2) the target in the second cross-linking step was the CE4 first cross-linked complex to obtain a CE4 second cross-linked complex.
3. Third cross-linking step: the third cross-linking step in CE4 was similar to that of Example 1, and the only difference was that the target in the third cross-linking step was the CE4 second cross-linked complex to obtain a CE4 cross-linked material.
4. Cross-linking neutralization step: the cross-linking neutralization step in CE4 was similar to that of Example 1, and the only difference was that the neutralization target was the CE4 cross-linked material to obtain a CE4 cross-linked product.
(V) Comparative example 5: 2 types of materials (collagen solution and collagen peptide solution) were subjected to a single cross-linking step with one cross-linking agent (GA). The details were as follows:

### 1. Cross-Linking Step:

(1) Mixing step: the collagen solution, the collagen peptide solution, the cross-linking agent (GA) and sodium hydroxide were added into PBS to obtain a CE5 mixture; wherein the CE5 mixture had a pH=10. Based on a total volume of the CE5 mixture, the collagen solution was in an amount of 20 w/v %, the collagen peptide solution was in an amount of 20 w/v %, and the cross-linking agent (GA) was in an amount of 0.01 w/v %. That is, the CE5 mixture was obtained after the mixing step.
(2) Cross-linking step: the CE5 mixture was stirred at a rotation speed of 250 rpm at room temperature for 24×3=72 hours to obtain a CE5 cross-linking solution.
(3) Precipitation step: the precipitation step in CE5 was similar to the first precipitation step in Example 1, and the only difference was that the precipitation target was the CE5 cross-linking solution to obtain a CE5 sediment.
(4) Cleaning step: the cleaning step in CE5 was similar to the first cleaning step in Example 1, and the only difference was that the cleaning target was the CE5 sediment to obtain a CE5 cross-linked complex.
2. Cross-linking neutralization step: the cross-linking neutralization step in CE5 was similar to that in Example 1, and the only difference was that the neutralization target was the CE5 cross-linked complex to obtain a CE5 cross-linked product.
(VI) Comparative example 6: 2 types of materials (first hyaluronic acid and second hyaluronic acid) were subjected to a single cross-linking step with one cross-linking agent (BDDE). The details were as follows:

### 1. Cross-Linking Step:

(1) Mixing step: the first hyaluronic acid, the second hyaluronic acid, the cross-linking agent (BDDE) and sodium hydroxide were added into PBS to obtain a CE6 mixture; wherein the CE6 mixture had a pH=10. Based on a total volume of the CE6 mixture, the first hyaluronic acid was in an amount of 1 w/v %, the second hyaluronic acid was in an amount of 1 w/v %, and the cross-linking agent (BDDE) was in an amount of 2 w/v %. That is, the CE6 mixture was obtained after the mixing step.
(2) Cross-linking step: the CE6 mixture was stirred at a rotation speed of 250 rpm at room temperature for 24×3=72 hours to obtain a CE6 cross-linking solution.
(3) Precipitation step: the precipitation step in CE6 was similar to the first precipitation step in Example 1, and the only difference was that the precipitation target was the CE6 cross-linking solution to obtain a CE6 sediment.
(4) Cleaning step: the cleaning step in CE6 was similar to the first cleaning step in Example 1, and the only difference was that the cleaning target was the CE6 sediment to obtain a CE6 cross-linked complex.
2. Cross-linking neutralization step: the cross-linking neutralization step in CE6 was similar to that in Example 1, and the only difference was that the neutralization target was the CE6 cross-linked complex to obtain a CE6 cross-linked product.
(VII) Comparative example 7: CE7 was CE5 further added with exosomes. The details were as follows: The preparation method of CE7 was similar to that of Example 2, and the only difference was that the target to be added with exosomes was the CE5 cross-linked product, not the biological cross-linked material in Example 1, to obtain a CE7 mixture. Further, the adipose-derived stem cell exosomes in the CE7 mixture were in a concentration of 1×10⁷ EV/ml which was the same as that in Example 2.
(VIII) Comparative example 8: CE8 was CE6 further added with exosomes. The details were as follows: The preparation method of CE8 was similar to that of Example 2, and the only difference was that the target to be added with exosomes was the CE6 cross-linked product, not the biological cross-linked material in Example 1, to obtain a CE8 mixture. Further, the adipose-derived stem cell exosomes in the CE8 mixture were in a concentration of 1×10⁷ EV/ml which was the same as that in Example 2.
(IX) Comparative example 9: the preparation method of CE9 was similar to that of CE3, and the only difference was the collagen source: the skins of pig and cattle at a weight ratio of 1:1.
(X) Comparative example 10: the preparation method of CE10 was similar to that of CE3, and the only difference was the collagen source: the skins of pig and goat at a weight ratio of 1:1.
(XI) Comparative example 11: the preparation method of CE11 was similar to that of CE3, and the only difference was the collagen source: the skins of cattle and goat at a weight ratio of 1:1

### Analysis I: Cross-Linking Degree, Collagen Degradation Rate and Hyaluronic Acid Degradation Rate

This analysis intended to know the structural strength of the biological cross-linked material product, and the comparisons of cross-linking degree, collagen degradation rate and hyaluronic acid degradation rate were carried out for Example 1, Example 3 to Example 5, Comparative example 1 to Comparative example 6, and Comparative example 9 to Comparative example 11. The details were as follows:

### (I) Cross-Linking Degree Analysis

TNBS analysis method: Trinitrobrnzen sulfonic acid (TNBS) can combine with the free amine groups in proteins to form a yellow substance, which can be quantified by using a spectrophotometer to measure the absorbance value at a wavelength of 345 nm. Therefore, the present invention used the TNBS method to detect free amine groups and calculate the cross-linking degree. The details were as follows:

### 1. Sample Preparation:

(1) Test sample: the biological cross-linked material products of all groups were respectively freeze-dried to obtain a respective lyophilized powder as a test sample.
(2) Control sample: the raw materials of all groups were respectively freeze-dried to obtain a respective lyophilized powder as a control sample. For example, A. the control sample for Example 1 was prepared by mixing 4 types of materials comprising collagen solution, collagen peptide solution, first hyaluronic acid and second hyaluronic acid at a weight ratio of 20:20:1:1 to obtain a material mixture, and the material mixture was freeze-dried to obtain a material lyophilized powder as the control sample for Example 1. B. the control sample for Comparative example 5 was prepared by mixing 2 types of materials comprising collagen solution and collagen peptide solution at a weight ratio of 20:20 to obtain a material mixture, and the material mixture was freeze-dried to obtain a material lyophilized powder as the control sample for Comparative example 5.

### 2. Analysis Steps:

(1) TNBS analysis method: 1 mg sample was added with a 100 μl 0.1% TNBS aqueous solution, placed in a dry bath for incubation at 50° C. for 1 hour, further added with a 300 μl 6N hydrochloric acid aqueous solution for incubation at 60° C. for 1.5 hours, and cooled to room temperature to obtain a test solution. 200 μl test solution was added to a 96-well plate, and the absorbance value thereof was measured by a spectrophotometer at a wavelength of 345 nm for quantification.
(2) Standard glycine solution: glycine has only one free amine group and serves as a reference standard. As the relationship between the absorbance value and the free amine group concentration may not be a linear relationship, standard glycine solutions with different concentrations were prepared and subjected to the TNBS analysis method and the measurement by a spectrophotometer at a wavelength of 345 nm to record the absorbance values for drawing a standard curve of the absorbance value against the concentration of glycine (concentration of free amine group concentration) for calculating the respective concentration of free amine group of the test sample and control sample in all groups.
(3) Calculation formula: Cross-linking degree (%)=[1−(The concentration of free amine group in Test sample/the concentration of free amine group in Control sample)]×100%. The concentration of free amine group in Test sample indicates the portion without cross-linking, and deducting such portion without cross-linking from all free amine groups (the concentration of free amine group in Control sample) can obtain the portion with cross-linking, which is the cross-linking degree.

### (II) Collagen Degradation Rate Analysis:

Ninhydrin analysis method: Ninhydrin can combine with the free amine groups in proteins to form a blue-violet substance, which can be quantified by using a spectrophotometer to measure the absorbance value at a wavelength of 570 nm. Therefore, the present invention used the Ninhydrin analysis method to detect the concentration of free amine group of the degraded collagen to calculate collagen degradation rate. The details were as follows:

### 1. Sample Preparation:

(1) Test sample: the biological cross-linked material product of all groups, not the lyophilized powders.
(2) Control sample: the material mixture (not lyophilized powders) of the raw materials in each group served as Control sample. For example, A. the control sample for Example 1 was prepared by mixing 4 types of materials comprising collagen solution, collagen peptide solution, first hyaluronic acid and second hyaluronic acid at a weight ratio of 20:20:1:1 to obtain a material mixture. B. the control sample for Comparative example 5 was prepared by mixing 2 types of materials comprising collagen solution and collagen peptide solution at a weight ratio of 20:20 to obtain a material mixture.

### 2. Analysis Step:

(1) Collagen degradation: 1 g sample was added with a 0.1 ml collagenase aqueous solution for incubation at 37° C. for 24 hours, and centrifugated at 10000 rpm for 10 minutes to obtain a supernatant; wherein the collagenase aqueous solution comprised 20 mM NaH₂PO₄·H₂O and 0.36 mM CaCl₂·2H₂O, pH=7.4, and the concentration of collagenase was 0.5 unit/ml.
(2) Ninhydrin analysis method: 0.2 ml supernatant was added with 25 μl Ninhydrin (concentration: 20 mg/ml, item number: 151173, purchased from Sigma-Aldrich), incubated at 100° C. for 20 minutes, cooled to room temperature, and subjected to the absorbance measurement by a spectrophotometer at a wavelength of 570 nm for quantification.
(3) Standard glycine solution: standard glycine solutions with different concentrations were prepared and subjected to the Ninhydrin analysis method and the measurement by a spectrophotometer at a wavelength of 570 nm to record the absorbance values for drawing a standard curve of the absorbance value against the concentration of glycine (concentration of free amine group) for calculating the respective concentration of free amine group of the test sample and control sample in all groups after collagenase digestion.
(4) Calculation formula: Collagen degradation rate (%)=(The concentration of free amine group in Test sample/the concentration of free amine group in Control sample)×100%.

### (III) Hyaluronic Acid Degradation Rate Analysis:

D-Glucurono-δ-lactone (D-GUL) is one of the main products after the degradation of hyaluronic acid and can be used to quantify the degradation rate of hyaluronic acid. Further, D-GUL will be degraded into D-Glucuronic Acid (D-GlcA) in an acidic environment. The carboxyl group (—COOH) in D-GlcA can undergo a condensation reaction with carbazole to produce a purple-red substance for quantification by measuring the absorbance values with a spectrophotometer at a wavelength of 530 nm. The details were as follows:

1. Sample preparation:
(1) Test sample: the biological cross-linked material product of all groups, not the lyophilized powders.
(2) Control sample: the material mixture (not lyophilized powders) of the raw materials in each group.

### 2. Analysis Step:

(1) Hyaluronic acid degradation: 1 g sample was added with 300 units hyaluronidase, further added with deionized water until the total volume reached 2 ml, incubated at 42° C. for 60 minutes, added with 3 ml anhydrous ethanol to stop enzymatic reaction, and centrifugated at 12000 rpm for 20 minutes to obtain a supernatant.
(2) Carbazole analysis method: experimental procedures thereof referred to the journal article: Liuyi Chang et al., Comparative Properties of Hyaluronic Acid Hydrogel Cross-linked with 1, 4-Butanediol Diglycidyl Ether Assayed Using a Marine Hyaluronidase, IOP Conference Series: Materials Science and Engineering, Volume 493, 2nd International Conference on Frontiers of Materials Synthesis and Processing 10-11 Nov. 2018, Sanya, China. That is, 100 μl supernatant was added with 200 μl 0.1 w/v % carbazole solution in the solvent of anhydrous ethanol, boiled at 100° C. for 10 minutes to develop color, and subjected to the absorbance measurement by a spectrophotometer at a wavelength of 530 nm for quantification.
(3) Standard D-Glucurono-δ-lactone (D-GUL) solution: standard D-GUL solutions with different concentrations were prepared, analyzed by the carbazole analysis method and measured by a spectrophotometer at a wavelength of 530 nm to record the absorbance values for drawing a standard curve of the absorbance value against the concentration of D-GUL for calculating the respective concentration of D-GUL of the test sample and control sample in all groups after hyaluronidase digestion.
(4) Calculation formula: hyaluronic acid degradation rate (%)=(The concentration of D-GUL in Test sample/The concentration of D-GUL in Control sample)×100%.

### (IV) Results

Each group performed three repeated tests, and the results of the cross-linking degree, collagen degradation rate and hyaluronic acid degradation rate of all groups were shown in Table 3.

TABLE 3
the cross-linking degree, collagen degradation rate and hyaluronic
acid degradation rate of all groups (comprising the respective
values of the three repeated tests and the average thereof)

| | Cross- | Collagen | Hyaluronic acid |
|---|---|---|---|
| Group | linking | degradation | degradation |
| No. | degree (%) | rate (%) | rate (%) |
| E1 | 75.62 | 9.17 | 12.52 |
| | 70.44 | 10.41 | 10.82 |
| | 79.2 | 8.34 | 15.77 |
| | average: 75.09 | average: 9.31 | average: 13.04 |
| E3 | 80 | 8.31 | 11.53 |
| | 75 | 7.26 | 14.23 |
| | 71 | 6.54 | 15.77 |
| | average: 75.33 | average: 7.37 | average: 13.84 |
| E4 | 65 | 8.32 | 16.22 |
| | 78 | 9.51 | 11.36 |
| | 80 | 8.24 | 13.5 |
| | average: 74.33 | average: 8.69 | average: 13.69 |
| E5 | 92 | 6.32 | 12.03 |
| | 65 | 4.32 | 12.58 |
| | 73 | 5.47 | 14.39 |
| | average: 76.67 | average: 5.37 | average: 13.00 |
| CE1 | 50.11 | 22 | 18.6 |
| | 45.77 | 25 | 22.5 |
| | 52.34 | 18.54 | 19.33 |
| | average: 49.41 | average: 21.85 | average: 20.14 |
| CE2 | 11.02 | 34.42 | 38.5 |
| | 10.12 | 40.51 | 42.6 |
| | 9.52 | 30.21 | 33.2 |
| | average: 10.22 | average: 35.05 | average: 38.10 |
| CE3 | 32.88 | 49.83 | 52.1 |
| | 36.51 | 54.89 | 57.8 |
| | 29.43 | 55.21 | 55.6 |
| | average: 32.94 | average: 53.31 | average: 55.17 |
| CE4 | 34.44 | 32.54 | 30.5 |
| | 40.2 | 29.11 | 28.7 |
| | 30.12 | 34.21 | 35.7 |
| | average: 34.92 | average: 31.95 | average: 31.63 |
| CE5 | N/A | 45.84 | N/A |
| | | 53.21 | |
| | | 40.38 | |
| | | average: 46.48 | |
| CE6 | N/A | N/A | 52.31 |
| | | | 40.33 |
| | | | 57.02 |
| | | | average: 49.89 |
| CE9 | 31.88 | 44.83 | 49.23 |
| | 37.51 | 50.89 | 45.39 |
| | 25.43 | 55.11 | 54.23 |
| | average: 31.61 | average: 50.28 | average: 49.62 |
| CE10 | 31.55 | 49.83 | 53.11 |
| | 30.25 | 45.31 | 58.21 |
| | 35.89 | 40.22 | 50.22 |
| | average: 32.56 | average: 45.12 | average: 53.85 |
| CE11 | 36.51 | 40.21 | 50.14 |
| | 29.87 | 54.89 | 49.25 |
| | 24.53 | 45.52 | 57.23 |
| | average: 30.30 | average: 46.87 | average: 52.21 |

According to Table 3, first, Example 1 and Example 3 to Example 5 all had (1) an average cross-linking degree greater than 74%, which indicated a high degree of cross-linking; (2) an average collagen degradation rate less than 10%, which indicated a low collagen degradation rate; and (3) an average hyaluronic acid degradation rate less than 14%, which indicated a low hyaluronic acid degradation rate.

In comparison, Comparative example 1 to Comparative example 6 and Comparative example 9 to Comparative example 11 all had (1) an average cross-linking degree less than 50%, which was significantly less than those (at least 74%) of Example 1 and Example 3 to Example 5; (2) an average collagen degradation rate greater than 20%, which was significantly greater than those (less than 10%) of Example 1 and Example 3 to Example 5; and (3) an average hyaluronic acid degradation rate greater than 20%, which was significantly greater than those (less than 14%) of Example 1 and Example 3 to Example 5. Accordingly, in comparison with Comparative example 1 to Comparative example 6 and Comparative example 9 to Comparative example 11, Example 1 and Example 3 to Example 5 were less easily degraded by collagenase and hyaluronidase in the human body, and can greatly increase the residence time of the biological cross-linked material in the human body to effectively reduce the frequency of supplementation of the biological cross-linked material.

Second, in the comparison among Example 1 and Example 3 to Example 5, both Example 3 and Example 5 (collagen source thereof comprising skin of cattle) had a collagen degradation rate less than those of Example 1 and Example 4, one may find that the skin of cattle as collagen source can further lower the collagen degradation rate to increase the residence time of the biological cross-linked material in the human body, and effectively reduce the frequency of supplementation of the biological cross-linked material.

Third, in the comparison of the three cross-linking steps in Example 1 and the single cross-linking step in Comparative example 1, (1) Example 1 had an average cross-linking degree (75.09%) significantly greater than that (49.41%) of Comparative example 1; (2) Example 1 had an average collagen degradation rate (9.31%) significantly less than that (21.85%) of Comparative example 1; and (3) Example 1 had an average hyaluronic acid degradation rate (13.04%) significantly less than that (20.14%) of Comparative example 1. Therefore, in comparison with a single cross-linking step, the present invention adopting three cross-linking steps can effectively increase the cross-linking degree and lower the collagen degradation rate and hyaluronic acid degradation rate to increase the residence time of the biological cross-linked material in the human body, and effectively reduce the frequency of supplementation of the biological cross-linked material.

Fourth, in the comparison of the sequential use of three different cross-linking agents (BDDE, GA and EDC) for the three cross-linking steps in Example 1 and the use of the single cross-linking agent for the three cross-linking steps in Comparative examples 2 to 4, (1) Example 1 had an average cross-linking degree (75.09%) significantly greater than those (34.92% for maximum) of Comparative examples 2 to 4; (2) Example 1 had an average collagen degradation rate (9.31%) significantly less than those (31.95% for minimum) of Comparative examples 2 to 4; and (3) Example 1 had an average hyaluronic acid degradation rate (13.04%) significantly less than those (31.63% for minimum) of Comparative examples 2 to 4. Therefore, in comparison with a single cross-linking agent, the sequential use of the three different cross-linking agents (BDDE, GA and EDC) for the three cross-linking steps of the present invention can effectively increase the cross-linking degree and lower the collagen degradation rate and hyaluronic acid degradation rate to increase the residence time of the biological cross-linked material in the human body, and effectively reduce the frequency of supplementation of the biological cross-linked material.

Fifth, Example 1 adopted 4 types of raw materials comprising the combination of collagen raw materials and hyaluronic acid raw materials, and (1) achieved an average collagen degradation rate (9.31%) significantly less than that (46.48%) of Comparative example 5 which adopted collagen raw materials only, and (2) achieved an average hyaluronic acid degradation rate (13.04%) significantly less than that (49.89%) of Comparative example 6 which adopted hyaluronic acid raw materials only. Therefore, in comparison with the use of single type of collagen raw materials or hyaluronic acid raw materials, the use of 4 types of raw materials comprising the combination of collagen raw materials and hyaluronic acid raw materials can effectively lower the collagen degradation rate and hyaluronic acid degradation rate to increase the residence time of the biological cross-linked material in the human body, and effectively reduce the frequency of supplementation of the biological cross-linked material.

### Analysis II: Yields

This analysis intended to know the yield of the biological cross-linked material product, and comprised 5 groups: Example 1 and Comparative example 1 to Comparative example 4. The details were as follows:

### (I) Yield Analysis

Calculation formula: Yield (%)=(the wet weight of biological cross-linked material product/the wet weight of raw material)×100%; wherein the wet weight of raw material was the total wet weight of the collagen solution, the collagen peptide solution, the first hyaluronic acid and the second hyaluronic acid, and did not include the weights of the cross-linking agents.

(II) Results: The Yields of all Groups were Shown in Table 4.

TABLE 4
The yields of all groups

| Group | E1 | CE1 | CE2 | CE3 | CE4 |
|---|---|---|---|---|---|
| Yields | 83.2 | 80.1 | 60.2 | 91.2 | 72.1 |
| (%) | 87.4 | 81.1 | 51.3 | 89.3 | 80 |
| | 82.2 | 75.3 | 63.31 | 94.1 | 65.2 |
| average | 84.27 | 78.83 | 58.27 | 91.53 | 72.43 |

According to Table 4, the yield of Comparative example 2 was significantly lower than the other groups which had similar yields. Further, the yield of Comparative example 3 was greater than that of Example 1, which indicated that more raw materials stayed in the biological cross-linked material product after the cross-linking step. However, according to Table 3, Comparative example 3 had a structural strength significantly less than Example 1, which indicated that Example 1 had a cross-linking quality significantly higher that of Comparative example 3.

### Analysis III: Cell Viability Analysis

This analysis intended to know the effects on cell viability by using the combination of the biological cross-linked material product and exosomes, and comprised: Blank groups, Control groups and Experimental groups. Further, Experimental groups comprised: (1) Example 2, (2) Example 2 after long-term storage, (3) Comparative example 7 and (4) Comparative example 8; wherein Example 2 after long-term storage indicated that the sample (Example 2) was kept in a refrigerated environment at 4° C. for 3 months before this analysis. All other groups were subjected to this analysis within 3 days after the samples were available. Further, both Blank groups and Control groups had the groups corresponding to those in Experimental groups. The details were as follows:

(I) Blank Groups: The Biological Cross-Linked Material Product was Used to Form a Coating without Addition of Cells:

Each Blank group used the biological cross-linked material product corresponding to those in the Experimental groups, and the steps were as follows: 0.1 g biological cross-linked material product was added to 24-well culture plate for 24 hours, added with and soaked in 0.5 ml cell culture medium for 24 hours to make the biological cross-linked material product attach to the bottom of the well to form a coating; wherein the cell culture medium comprised Dulbecco's Modified Eagle Medium (DMEM) with 10 volume percent of Fetal Bovine Serum (FBS).

The cell culture medium was removed, added with 1 ml cell culture medium (without cells) for each well, transferred to a cell culture incubator to incubate for 2 hours in a constant environment of 37° C. and 5% carbon dioxide. Each blank group was further added with 0.5 ml cell culture medium (without cells) to incubate for 7 days, replaced with 0.5 ml fresh cell culture medium (without cells), subjected to the experiment steps according to the instruction of kit (Cell Counting Kit-8, CCK-8, purchased from Sigma-Aldrich/Merck). 100 μl from each blank group was transferred to a 96-well culture plate to measure the absorbance value by a spectrophotometer at a wavelength of 450 nm.

(II) Control Groups: No Coating Formed by the Biological Cross-Linked Material Product, and Cells were Provided:

1 ml cell culture medium with cells (the cell culture medium in blank group and 1×10⁴ human dermal fibroblast (HDF)) was provided to each well in a 24-well culture plate, and transferred to a cell culture incubator to incubate for 7 days in a constant environment of 37° C. and 5% carbon dioxide, replaced with 0.5 ml fresh cell culture medium, and subjected to the experiment steps according to the instruction of kit (Cell Counting Kit-8). 100 μl from each control group was transferred to a 96-well culture plate to measure the absorbance value by a spectrophotometer at a wavelength of 450 nm.

### (III) Experimental Groups:

0.1 g biological cross-linked material product was added to each well of a 24-well culture plate for 24 hours, added with and soaked in 0.5 ml cell culture medium for 24 hours to make the biological cross-linked material product attach to the bottom of the well to form a coating. 1 ml cell culture medium with cells (the same as those in the control group) was provided to each well in a 24-well culture plate, and transferred to a cell culture incubator to incubate for 7 days in a constant environment of 37° C. and 5% carbon dioxide, replaced with 0.5 ml fresh cell culture medium, and subjected to the experiment steps according to the instruction of kit (Cell Counting Kit-8). 100 μl from each Experimental group was transferred to a 96-well culture plate to measure the absorbance value by a spectrophotometer at a wavelength of 450 nm.

### (IV) Calculation Formula:

Cell viability (%)=(the absorbance value of Experimental group−the absorbance value of Blank group)/(the absorbance value of Control group−the absorbance value of Blank group)×100%.

### (V) Results: The Cell Viability of all Experimental Groups was Shown in Table 5.

TABLE 5
the cell viability of all Experimental groups

| | E2 | E2 after long-term storage | CE7 | CE8 |
|---|---|---|---|---|
| Cell viability | 240 | 248 | 170 | 224 |
| (%) | 251 | 230 | 182 | 180 |
| | 238 | 223 | 160 | 190 |
| Average | 243 | 233.67 | 170.67 | 198.00 |
| (%) | | | | |

According to Table 5, E2 and E2 after long-term storage achieved similar cell viability, so the biological cross-linked material product of Example 2 demonstrated excellent stability even after 3 months storage. Further, Example 2 had a cell viability significantly greater than those of Comparative example 7 and Comparative example 8. Therefore, the biological cross-linked material product of Example 2 can effectively promote the growth of human skin fibroblasts and skin health after injection into the skin.

To sum up, the biological mixture, the biological cross-linked material or the biological cross-linked material product of the present invention demonstrates an excellent structural strength, such as a high degree of cross-linking, low collagen degradation rate and low hyaluronic acid degradation rate. Further, the combination of the biological mixture, the biological cross-linked material of the present invention or biological cross-linked material product with exosomes can further maintain skin health effectively.

What is claimed is:

1. A biological mixture, comprising a biological cross-linked material, and the biological cross-linked material comprising collagen, collagen peptide, a first hyaluronic acid and a second hyaluronic acid;
wherein the first hyaluronic acid has a molecular weight of 100 kilodaltons (kDa) to 500 kDa, and the second hyaluronic acid has a molecular weight of 1500 kDa to 3000 kDa;
the collagen, the collagen peptide, the first hyaluronic acid and the second hyaluronic acid are cross-linked with each other; and
the biological cross-linked material has an average degree of cross-linking from 65% to 97%.

2. The biological mixture as claimed in claim 1, wherein based on a total volume of the biological cross-linked material, the collagen and the collagen peptide are in a total amount of 30 mg/ml to 80 mg/ml.

3. The biological mixture as claimed in claim 1, wherein based on a total volume of the biological cross-linked material, the first hyaluronic acid and the second hyaluronic acid are in a total amount of 1 mg/ml to 18 mg/ml.

4. The biological mixture as claimed in claim 1, further comprising exosomes.

5. The biological mixture as claimed in claim 4, wherein based on a total volume of the biological mixture, the exosomes have a concentration of 1×10⁵ extracellular vesicles (EV)/ml to 1×10¹¹ EV/ml.

6. A method for skin tissue filling, comprising providing the biological mixture as claimed in claim 1 into a skin.

7. A method for skin tissue filling, comprising providing the biological mixture as claimed in claim 2 into a skin.

8. A method for skin tissue filling, comprising providing the biological mixture as claimed in claim 3 into a skin.

9. A method for skin tissue filling, comprising providing the biological mixture as claimed in claim 4 into a skin.

10. A method for skin tissue filling, comprising providing the biological mixture as claimed in claim 5 into a skin.

11. The method as claimed in claim 6, wherein the biological mixture is in an injection dosage form.

12. The method as claimed in claim 7, wherein the biological mixture is in an injection dosage form.

13. The method as claimed in claim 8, wherein the biological mixture is in an injection dosage form.

14. The method as claimed in claim 9, wherein the biological mixture is in an injection dosage form.

15. The method as claimed in claim 10, wherein the biological mixture is in an injection dosage form.

16. A preparation method for the biological mixture as claimed in claim 1, comprising:
a first cross-linking step: mixing the collagen, the first hyaluronic acid, a first cross-linking agent and a first carrier to obtain a first mixture for carrying out a first cross-linking reaction for 20 hours to 28 hours to obtain a first cross-linked complex; wherein based on a total volume of the first mixture, the collagen is in an amount of 10 weight/volume percent (w/v %) to 40 w/v %, and the first hyaluronic acid is in an amount of 0.1 w/v % to 9 w/v %;
a second cross-linking step: mixing the first cross-linked complex, the second hyaluronic acid, a second cross-linking agent and a second carrier to obtain a second mixture for carrying out a second cross-linking reaction for 20 hours to 28 hours to obtain a second cross-linked complex; wherein based on a total volume of the second mixture, the first cross-linked complex is in an amount of 10 w/v % to 40 w/v %, and the second hyaluronic acid is in an amount of 0.1 w/v % to 9 w/v %; and
a third cross-linking step: mixing the second cross-linked complex, the collagen peptide, a third cross-linking agent and a third carrier to obtain a third mixture for carrying out a third cross-linking reaction for 20 hours to 28 hours to obtain the biological cross-linked material; wherein based on a total volume of the third mixture, the second cross-linked complex is in an amount of 10 w/v % to 40 w/v %, and the collagen peptide is in an amount of 10 w/v % to 40 w/v %; wherein
the first cross-linking agent, the second cross-linking agent and the third cross-linking agent are different from each other, and the first carrier, the second carrier and the third carrier each comprise water.

17. The preparation method as claimed in claim 16, wherein the first cross-linking agent comprises 1,4-butanediol diglycidal ether, and based on a total volume of the first mixture, the first cross-linking agent is in an amount of 0.5 w/v % to 5 w/v %;
the second cross-linking agent comprises glutaraldehyde, and based on a total volume of the second mixture, the second cross-linking agent is in an amount of 0.005 w/v % to 0.1 w/v %; and
the third cross-linking agent comprises 1-ethyl-3-[3-dimethylaminopropyl]carbodiimide, and based on a total volume of the third mixture, the third cross-linking agent is in an amount of 0.5 w/v % to 5 w/v %.

18. The preparation method as claimed in claim 16, wherein the first cross-linking step and/or the third cross-linking step comprises adding an alkaline agent.
